(19) United States
(12) Patent Application Publication
(10) Pub. No.: US 2026/0185986 A1
(43) Pub. Date: Jul. 2, 2026

(54) METHODS AND SYSTEMS FOR DETECTING AN ANALYTE WITH A BIOMOLECULE

(71) Applicant: TELEDYNE FLIR DEFENSE, INC., North Billerica, MA (US)

(72) Inventors: Jennifer L. Poole, Delmont, PA (US); Miranda Jacobs, Pittsburgh, PA (US); Ann Elizabeth Donnelly, Pittsburgh, PA (US)

(21) Appl. No.: 19/430,708

(22) Filed: Dec. 23, 2025

Related U.S. Application Data
(60) Provisional application No. 63/739,360, filed on Dec. 27, 2024.

(51) Int. Cl. G01N 33/536; G01N 33/68

(52) U.S. Cl. CPC G01N 33/536; G01N 33/68

(57) ABSTRACT

Methods and systems for detecting an analyte with a biomolecule are provided. The method comprises introducing a biomolecule to the object, introducing a substrate to the object, and introducing a solvent to the object. The substrate, biomolecule, and solvent form a mixture on the object. The biomolecule comprises a recognition portion capable to bind with the analyte and a trigger portion capable to participate in an amplifying cascade chemical reaction responsive to the analyte binding with the recognition portion. The method comprises binding the recognition portion of the biomolecule with the analyte. The method comprises chemically reacting the trigger portion of the biomolecule with the substrate via an amplifying cascade chemical reaction to produce an amplified product responsive to the analyte binding with the recognition portion. The method comprises detecting the amplified product, thereby detecting presence of the analyte.

### CROSS-REFERENCE TO RELATED APPLICATION

This application claims the benefit of and priority under 35 U.S.C. § 119 (e) to U.S. Provisional Patent Application Ser. No. 63/739,360, filed Dec. 27, 2024, entitled “METHODS AND SYSTEMS FOR DETECTING AN ANALYTE WITH A BIOMOLECULE,” the contents of which is hereby incorporated by reference in its entirety herein.

### BACKGROUND

Existing technologies to detect biological contamination on a surface commonly rely on collection of discrete sampling followed by time consuming and complex sample interrogation at a laboratory facility. There are challenges with detecting biological contamination.

### SUMMARY

According to one non-limiting aspect of the present disclosure, a method for detecting an analyte on an object is provided. The method comprises introducing a biomolecule to the object, introducing a substrate to the object, and introducing a solvent to the object. The substrate, biomolecule, and solvent form a mixture on the object. The biomolecule comprises a recognition portion capable to bind with the analyte and a trigger portion capable to participate in an amplifying cascade chemical reaction responsive to the analyte binding with the recognition portion. The method comprises binding the recognition portion of the biomolecule with the analyte. The method comprises chemically reacting the trigger portion of the biomolecule with the substrate via an amplifying cascade chemical reaction to produce an amplified product responsive to the analyte binding with the recognition portion. The method comprises detecting the amplified product, thereby detecting presence of the analyte.

According to another non-limiting aspect of the present disclosure, a biomolecule for detecting an analyte is provided. The biomolecule comprises a recognition portion capable to bind with the analyte and a trigger portion capable to participate in a amplifying cascade chemical reaction responsive to the analyte binding with the recognition portion.

According to another non-limiting aspect of the present disclosure, a system for detecting an analyte is provided. The system comprises a biomolecule for detecting an analyte and a substrate. The biomolecule comprises a recognition portion capable to bind with the analyte and a trigger portion capable to participate in a amplifying cascade chemical reaction responsive to the analyte binding with the recognition portion. The substrate is capable to react with the trigger portion of the biomolecule to produce an amplifying cascade chemical reaction responsive to an analyte binding to the recognition portion.

It will be understood that the inventions disclosed and described in this specification are not limited to the aspects summarized in this Summary. The reader will appreciate the foregoing details, as well as others, upon considering the following detailed description of various non-limiting and non-exhaustive aspects according to this specification.

### BRIEF DESCRIPTION OF THE DRAWINGS

Features and advantages of the examples presented herein, and the manner of attaining them, will become more apparent, and the examples will be better understood, by reference to the following description taken in conjunction with the accompanying drawings, wherein:

FIG. 1 is a flow chart of a non-limiting embodiment of a method to detect an analyte with a biomolecule according to the present disclosure;

FIG. 2 is a diagram of a method to detect an analyte with a biomolecule according to Example 6;

FIG. 3 is a diagram of a method to detect an analyte with a biomolecule according to Example 7;

FIG. 4A is a structure of an immolative aryl azide PNA according to Example 7;

FIG. 4B is a structure of a phosphine PNA according to Example 7;

FIG. 4C is a structure of an autocatalytic acid amplification reagent according to Example 7;

FIG. 4D is a diagram of a method to detect an analyte with a biomolecule according to Example 7;

FIG. 5 is a diagram of a method to detect an analyte with a biomolecule via a Di-Part Protein Compliment Assay (PCA) according to Example 9;

FIG. 6 is a diagram of a method to detect an analyte with a biomolecule via a Tri-Part PCA according to Example 9;

FIG. 7 is a diagram of a method to detect an analyte with a biomolecule according to Example 10;

FIG. 8 is a diagram of a method to detect an analyte with a biomolecule according to Example 11; and

FIG. 9 is a diagram of a method to detect an analyte with a biomolecule according to Example 12.

If present, corresponding reference characters indicate corresponding parts throughout the several views. The exemplifications set out herein illustrate certain embodiments, in one form, and such exemplifications are not to be construed as limiting the scope of the appended claims in any manner.

### DETAILED DESCRIPTION OF NON-LIMITING EMBODIMENTS

Various examples are described and illustrated herein to provide an overall understanding of the structure, function, and use of the disclosed methods and systems. The various examples described and illustrated herein are non-limiting and non-exhaustive. Thus, the invention is not limited by the description of the various non-limiting and non-exhaustive examples disclosed herein. Rather, the invention is defined solely by the claims. The features and characteristics illustrated and/or described in connection with various examples may be combined with the features and characteristics of other examples. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be present in the prior art. The various embodiments disclosed and described in this specification can comprise, consist of, or consist essentially of the features and characteristics as variously described herein.

Any references herein to “various embodiments”, “some embodiments”, “one embodiment”, “an embodiment”, or like phrases mean that a particular feature, structure, or characteristic described in connection with an example is included in at least one embodiment. Thus, appearances of the phrases “in various embodiments”, “in some embodiments”, “in one embodiment”, “in an embodiment”, or like phrases in the specification do not necessarily refer to the same embodiment. Furthermore, the particular described features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation. Such modifications and variations are intended to be included within the scope of the present embodiments.

Use of existing biological detection technologies may be logistically burdensome and time consuming. The existing technologies may not be capable of visualizing and mapping biological contamination in a wash-free manner, directly on surfaces in the environment, and/or desirably within a well of a test plate. Existing technologies commonly rely on collection of discrete sampling followed by time consuming and complex sample interrogation at a laboratory facility. This can require the collection, logging, and scientific analysis of a large number of samples, which can induce a significant logistical burden, while providing a low level of confidence whether or not biological contamination was present.

Typically, physiological symptoms that result from exposure to biological contamination are not exhibited instantaneously and may be delayed and take significant time to manifest. This delay can considerably magnify the consequences of biological contamination through continued contact with and spreading of the biological contamination. The present inventors recognized a need to enhance the confidence level of detection of biological contamination, while also reducing logistical burden and enhancing response time.

The present disclosure provides methods and systems for detecting an analyte with a biomolecule that can enhance the confidence level of detection, reduce logistical burden, enhance response time, resist interferents, enhance sensitivity, and/or enhance storage stability. In various non-limiting embodiments, the methods and systems according to the present disclosure can be targeted for direct, single-step detection in real-time and/or can enable decontamination assurance for confidence that treated areas are clean (i.e., uncontaminated with the analyte).

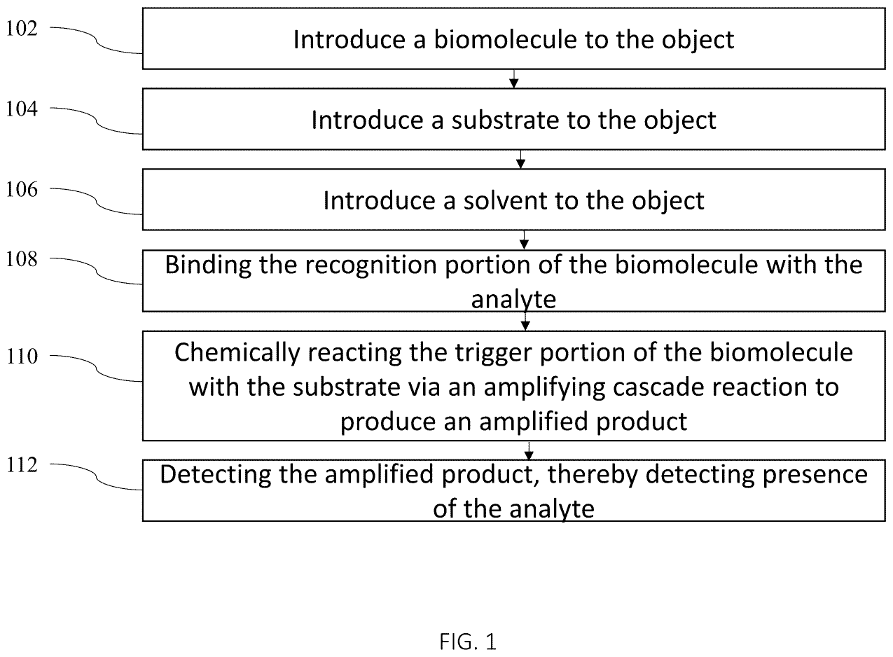

FIG. 1 provides an embodiment of a method according to the present disclosure for detecting an analyte on an object. The method can enhance the confidence level of detection, reduce logistical burden, enhance response time, enhance resistance to interferents, enhance sensitivity, and/or enhance storage stability relative to certain known methods.

With respect to FIG. 1, the method can be performed in, for example, a lab and/or on-site. Various embodiments of the method can be used for surface mapping of contamination, confirming decontamination is performed, and/or for sample testing in a lab.

With respect to FIG. 1, the object can comprise, for example, a vessel, a device, and/or other object. In various non-limiting embodiments, the object can comprise a surface of a device, a surface of a vessel (e.g., a sample well), and/or another surface. In certain non-limiting embodiments, the object can be a well of a plate and the method can be conducted within an analytical instrument, such as, for example, a Fourier Transform Infrared Spectroscopy (FTIR) apparatus, a spectrometer, a camera, or other instrument.

With respect to FIG. 1, the analyte can be a target molecule to be detected by the method. For example, the analyte can comprise contamination, such as, for example, biological contamination or other contamination. Biological contamination, as opposed to chemical warfare agents, can be difficult to detect by previous techniques as biological contamination may be unreactive to typical chemistries and/or be unable to significantly produce sufficient signal for measurement. Previous molecules that could bind to biological contamination may not be able to transduce and amplify a signal derived from the binding event. In various non-limiting embodiments, the analyte can comprise a nucleotide, a peptide, a protein, a glycan, a lipid, a glycoprotein, a bacteria, a virus, a fungus, a mammalian biomolecule, a drug, a synthetic polymer, and/or other analyte. In various non-limiting embodiments, the analyte can comprise a spore, a virus, a drug (e.g., an opioid), a bacteria, and/or other analyte.

Again referring to FIG. 1, the method comprises introducing a biomolecule to the object at step 102, introducing a substrate to the object at step 104, and introducing a solvent to the object at step 106. The substrate, biomolecule, and solvent can form a mixture on the object.

Each or two of Steps 102, 104, and 106 may occur simultaneously or the steps may occur sequentially, and the steps may occur in any order. For example, the mixture may be created prior to, during, and/or after introduction of the biomolecule, the substrate, and the solvent to the object. For example, the components of a system can be mixed prior to introduction to form the mixture, and the mixture can be introduced to the object. In certain non-limiting embodiments, less than all of the components of the system can be introduced to the object at the same time.

In certain non-limiting embodiments, the mixture can comprise a ratio of molecules of reporters to molecules of biomolecules of at least 1, such as, for example, at least 10, at least 100, at least 500, or at least 1000.

The components (e.g., biomolecule, solvent, substrate) of the system of FIG. 1 can comprise various forms. For example, each of the components can individually be a powder (e.g., lyophilized powder), a liquid, or a combination of powder and liquid. For example, the biomolecule can be a lyophilized powder and can be mixed with the solvent prior to introduction to the object. The substrate may or may not be mixed with the biomolecule and the solvent prior to introduction to the object.

The components can be introduced to the object by deposition, flowing, pipetting, spraying, dipping, roll coating, flow coating, and/or film coating. In various non-limiting embodiments, the components can be sprayed onto a surface of the object. In certain non-limiting embodiments, the components can be introduced to a well of a sample plate by pipetting.

In various non-limiting embodiments, other components can be introduced to the object and be a component of the mixture. For example, a reporter, a light scattering additive, an adhesive polymer, a protein stabilizer, a protein stabilizing sugar, a surfactant, a buffer (e.g., pH stabilizing salt), a reaction substrate, a cofactor, a solubilizing agent, a co-protein, an enzyme, a co-substrate, and/or an additional solvent can be introduced to the object.

The biomolecule can comprise a nucleotide-type reporter, a protein-type reporter, and/or a receptor-type reporter. In various non-limiting embodiments, the protein-type reporter can comprise a nanobody, a single-domain antibody, a glycan-binding protein, a lipid-binding protein, a protein-protein interaction, a de novo designed binding protein, and/or other protein-type reporters. The biomolecule can be capable to identify and bind to the analyte and initiate an amplifying cascade chemical reaction.

As used herein, an “amplifying cascade reaction” is a series of events (e.g. chemical reaction, binding event) that create and/or activate multiple downstream components in succession converting a small event into a larger signal that can facilitate detection of the analyte. For example, an initial event triggers an initial reaction which results in the generation of multiple second reactions, each of which initiates multiple third reactions, etc, wherein the ‘signal’ produced from the final reaction is amplified from the initial stimulus.

The biomolecule of the method of FIG. 1 can comprise a recognition portion and a trigger portion. The recognition portion and the trigger portion can be operatively coupled to one another. For example, the recognition portion and the trigger portion can be attached by at least one of a covalent bond, a hydrogen bonding, a Van Der Waals force, and a polymer. The recognition portion and the trigger portion can be in functional communication with one another to facilitate detection of the analyte. For example, the recognition portion can communicate to the trigger portion that binding of the analyte occurred through binding, a chemical reaction, and/or confirmational change.

The recognition portion of the biomolecule can be capable to identify the analyte. The recognition portion can be capable to bind with the analyte and/or otherwise associate with the analyte. The binding and/or association with the analyte can facilitate activation of the trigger portion.

In various non-limiting embodiments, the recognition portion can comprise at least one element selected from the group consisting of a receptor, an antibody, an aptamer, an antigen, a bacterial protein, a nanobody, a single-domain antibody, a glycan-binding protein (e.g., GBPs, lectins), a lipid-binding protein, a protein-protein interaction, a de novo designed binding protein, and a synthetic polymer (e.g., molecularly imprinted polymers (MIPs)).

Aptamers can be, for example, short (e.g., less than 100 nucleotides) single-stranded DNA or RNA molecules. Aptamers can have high binding affinity and selectivity for specific analytes. Aptamers can maintain the general features of oligonucleotides, including ease of production, Watson-Crick base pairing, predictable secondary and tertiary structures, high chemical and thermal stability, and/or site-specific modification of various functional groups.

Aptamer ligands can be generated through in vitro evolution techniques such as, for example, systematic evolution of ligands by exponential enrichment (SELEX), cell-SELEX, or systematic evolution of ligands by competitive selection (SELCOS), among other techniques. Aptamers can be produced in large quantities through chemical synthesis or biological production. Aptamers can be engineered to enhance target selectivity and avoid cross-reactivity with both matrix components and closely related biological interferents through the use of negative (counter) selections during the aptamer evolution process. Use of these selection techniques can discriminate between a desired target analyte and other closely related species with high resolution.

The trigger portion of the biomolecule can be capable to participate in an amplifying cascade chemical reaction responsive to the analyte binding with the recognition portion. The trigger portion can be in a first state of reduced, if any, chemical activity with a substrate prior to the recognition portion binding with the analyte. For example, the trigger portion can be autoinhibited and/or reduced in activity based on limited proximity to other trigger portions.

After the recognition portion binds to the analyte, the trigger portion can be activated and transform into a second state capable of increased chemical activity with the substrate relative to the first state. For example, in certain non-limiting embodiments, the trigger portion may be autoinhibited in the first state, and in the second state the trigger portion can undergo a structure change resulting in reduction in the autoinhibition. In various non-limiting embodiments, the biomolecule may be at a sufficiently low concentration on/in the object such that the biomolecules do not substantially aggregate and the trigger portions on each biomolecule do not substantially interact in a first state. The binding of the recognition portions to the analyte can aggregate the trigger portions together into close proximity, thereby increasing the local concentration, such that the trigger portions interact with one another to activate and transform into the second state.

In various non-limiting embodiments, the trigger portion can comprise at least a portion of at least one element selected from the group consisting of an enzyme, a DNAzyme, a self-amplifying molecular assembly, and a self-immolative polymer. For example, DNAzyme can self-recruit other DNAzymes and together participate in an amplifying cascade chemical reaction. In various non-limiting embodiments, DNAzyme can recruit a fluorescent protein to participate in an amplifying cascade chemical reaction, resulting in a fluorescent output.

In certain non-limiting embodiments, the trigger portion can be a portion of a split-enzyme. The trigger portion can activate by binding with another trigger portion to form a whole enzyme.

In various non-limiting embodiments, the trigger portion can be a proximity induced trigger portion. The trigger portions can be brought in proximity to one another by templating on DNA and/or an antibody-type recognition event. In certain non-limiting embodiments, the trigger portion can comprise a portion of an immolative polymer-type reporter system.

In certain non-limiting embodiments in which the recognition portion comprises an aptamer, the binding of aptamer to an analyte can depend on the secondary and tertiary structure of the aptamer. The use of these structural elements can be leveraged to create a manufacturable, modular sensor. The incorporation of a trigger portion comprising a structure-switching mechanism within the aptamer binding motif can facilitate the direct transduction of analyte identification events into signaling reporters.

Use of such a transduction element can simplify manufacturing of the system by reducing, if not eliminating, chemical conjugation of a secondary reporter to the recognition element. This mechanism permits the reporter signaling to be agnostic to the analyte and can increase the utility of the sensing mechanism as a platform which may be easily transferrable to future analytes with minimal system-level modification. Structure-switching features can be directly embedded into the biomolecule and optimized through the SELEX (Systematic Evolution of Ligands by Exponential Enrichment) process.

The substrate can be various molecules that are capable of participating in an amplifying cascade reaction with the trigger portion of the biomolecule. For example, the substrate can be an additional portion of a DNAzyme, hemin, an additional immolative polymer, an enzyme substrate, and/or a cofactor. In various non-limiting embodiments, the ratio of substrates to analyte binding events can be at least 2:1, at least 3:1, at least 4:1, at least 5:1, at least 10:1, at least 100:1, at least 500:1, at least 1,000:1, at least 10,000:1, at least 50,000:1, or at least 100,000:1.

Further referring to FIG. 1, the solvent can be an aqueous based solvent. For example, the solvent can comprise water and/or a buffer. In certain non-limiting embodiments, the solvent can comprise organic components, in addition to water. For example, the solvent can comprise xylene, toluene, hexane, heptane, octane, acetone, ethanol, methanol, and/or dimethyl sulfoxide. The solvent can be formulated to be compatible with the biomolecule and/or analyte.

The reporter can be a pH responsive molecule and/or an oxidative responsive molecule. In certain non-limiting embodiments, a reporter is not used and a parameter of the mixture can be measured directly. The reporter can be visual, fluorescent, chemiluminescent, UV, and/or IR based. In various non-limiting embodiments, the reporter can comprise cresol red, phenol red, metacresol purple, neutral red, curcumin, carboxy-semi naphthorhodaflur (carboxy-SNARF), fluorescein, luminol, oxatane, guaiacol, 4-aminoantipyrine, and/or 3-(N-Ethyl-3-methylanilino)-2-hydroxypropanesulfonic acid (EHSPT).

In certain non-limiting embodiments in which a light scattering additive is present, the light scattering additive can comprise, for example, a water insoluble particulate metal oxide. For example, the light scattering additive can comprise aluminum oxide, titanium dioxide, and/or a silica-coated titanium dioxide.

In certain non-limiting embodiments in which an adhesive polymer is present, the adhesive polymer can comprise, for example, poly(vinyl alcohol), carboxymethyl cellulose, and/or hydroxypropylmethyl cellulose.

In certain non-limiting embodiments in which a protein stabilizer is present, the protein stabilizer can comprise, for example, poly(ethylene glycol), poly(N-vinyl pyrrolidone), dextran, poly(vinyl alcohol), and/or poly(ethylene imine).

In certain non-limiting embodiments in which a protein stabilizing sugar is present, the protein stabilizing sugar can comprise, for example, sucrose, trehalose, mannitol, xylitol, maltose, dextrose, and/or glucose.

In certain non-limiting embodiments in which a surfactant is present, the surfactant can comprise, for example, polyethylene glycol p-(1,1,3,3-tetramethylbutyl)-phenyl ether (Triton X-100, Dow Chemical Company), polyoxyethylene (20) sorbitan monooleate (Tween-80, ICI Americas, Inc), poly oxyethylene (20) sorbitan monolaureate (Tween-20, ICI Americas, Inc.), polyoxyethylene lauryl ether (Brij-35, Caledon Laboratories Ltd.), lauramine oxide (AO-12, Mason Chemical Company), and/or sodium di-2-ethyl-hexyl Sulphosuccinate (AOT).

In certain non-limiting embodiments in which an additional solvent is present, the additional solvent can comprise, for example, xylene, toluene, hexane, heptane, octane, acetone, ethanol, and methanol.

At step 108, the method illustrated in FIG. 1 comprises binding the recognition portion of the biomolecule with the analyte. Binding can comprise, for example, forming an ionic bond, a covalent bond, a hydrogen bonding, a Van Der Waals interaction, and/or a conformationally induced interaction (e.g., lock and key fit) between the recognition portion of the biomolecule and the analyte. In various non-limiting embodiments, the binding associates the recognition portion with the analyte.

Binding the recognition portion of the biomolecule with the analyte can comprise, for example, activating the trigger portion such that the trigger portion can be transformed from a first state to a second state. For example, the trigger portion can be activated by proximity-based association with another trigger portion of a different biomolecule and/or reduction of autoinhibition of the trigger portion. The activation of the trigger portion can enable the trigger portion to participate in an amplifying cascade chemical reaction.

In various non-limiting embodiments, activating the trigger portion can comprise aggregating the biomolecules to the analyte such that a local concentration of the biomolecules increases and such that trigger portions from different biomolecules interact with one another. In certain non-limiting embodiments, activating the trigger portion can comprise structurally changing the trigger portion to reduce autoinhibition and thereby increase the activity of the trigger portion with the substrate.

At step 110, the method illustrated in FIG. 1 comprises chemically reacting the trigger portion of the biomolecule with the substrate via an amplifying cascade reaction to produce an amplified product responsive to the analyte binding with the recognition portion. The amplifying cascade reaction can enhance response time and/or enhance sensitivity. For example, in various non-limiting embodiments, the amplifying cascade reaction can exponentially amplify a reporter signal (e.g., an amplified product) upon analyte recognition to trigger a reporter signal cascade with components already existing in the mixture. The amplifying cascade reaction may not be dependent on in situ production of cascade components.

At step 112, the method illustrated in FIG. 1 comprises detecting the amplified product, thereby detecting presence of the analyte. In various non-limiting embodiments, detecting the amplified product can comprise measuring at least one parameter of the mixture. The parameter can comprise, for example, an absorbance, a transmittance, a color, an oxidative potential, a pH, light rotation, refractive index, Raman scattering, chemiluminescence, temperature, and/or volatile organic chemical content. The measurement may be performed, for example, visually (e.g., by color) and/or the measurement may be performed with an analytical instrument.

The detection of the amplified product may result in a binary present or not present determination of the amplified product and thus, a binary present or not present determination for the analyte. In various non-limiting embodiments, the detection of the amplified product may result in a determination of a degree of presence of the amplified product (e.g., a concentration) and thus, a determination of a degree of presence of the analyte.

In various non-limiting embodiments, step 112 can occur in a well-mixed solution in a vessel and the determination can be for the entire mixture.

In certain non-limiting embodiments, step 112 can occur locally within the mixture on the object. For example, steps 108 and 110 may not occur throughout the entire mixture and may only occur in localized contaminated regions on a surface of the object. The detection at step 112 can differentiate contaminated regions on the surface of the object from non-contaminated regions. Decontamination and/or further analysis of the surface of the object may then be focused on the contaminated regions. Real-time visualization of contaminated regions on surfaces can be directly used for mapping and/or tracking the location of the contaminated regions for decontamination assurance efforts.

Previous techniques typically utilize washing steps to remove non-specifically bound analyte to improve signal resolution. During the method according to the present disclosure, unbound biomolecule may not be removed from the object prior to chemically reacting the biomolecule with the substrate, which can enhance the efficiency of the method and/or reduce operator error. For example, the method according to the present disclosure may be a wash-free assay. In various non-limiting embodiments, as the biomolecule comprises both a recognition portion and a trigger portion, the biomolecule can be applied in a one-pot, add-and-read solution. In various non-limiting embodiments, steps 108, 110, and 112 can occur in-situ on and/or in the object.

Previous wash-free assays typically require sophisticated equipment, labeled ligands, or operate in a “turn off” fashion. The use of sophisticated equipment can impose limitations of time and cost, and restricts locations at which the assay may be performed. The use of labeled ligands unavoidably alters the binding kinetics of the ligands to be probed. Turn off assays are inherently less desirable than turn on assays because the lack of a reporter signal may not always be a positive indication of no contamination. In various non-limiting embodiments, the method of the present disclosure may not require sophisticated equipment, may not require labeled ligands, and/or may operate in a turn on fashion.

Previous binding assays use stoichiometric reporters. This restricts the sensitivity of the assay since one binding event will only ever provide one reporting event which can significantly limits the signal output. By utilizing an amplifying cascade reaction, greater reporter signal may be obtained from each binding event through continual generation of the reporter signal from the trigger portion.

Decoupling identification of the analyte and binding to the analyte at step 108 from the amplifying cascade reaction at step 110 can simplify assay expansion by accommodating new analytes through redesign of only the recognition portion. Embodiments of the method may also be multiplexed to detect multiple analytes by using different biomolecules having different recognition portions and different trigger portions on each. For example, multiple variants of luciferase exist, each of which produces a different wavelength of light. Different variants of luciferase could be used in tandem on different biomolecules to differentiate between analytes of interest, or report on more than one analyte at a time, all in a single “add and read”-type reaction.

Additional amplification of the report can occur through paired enzyme systems where the first enzyme generates the substrate for the second and/or further chemical reactions like immolative decomposition.

Embodiments of a system according to the present disclosure for detection of the analyte can comprise the biomolecule and the substrate and optionally other components, such as, for example, a solvent, a reporter, a light scattering additive, an adhesive polymer, a protein stabilizer, a protein stabilizing sugar, a surfactant, and/or an additional solvent. Each component can be provided as liquid and/or solid that is capable of being dissolved and/or dispersed into water as desired. In certain embodiment, the solid can comprise a lyophilized powder. In various non-limiting embodiments, a sufficient amount of solvent can be used to reconstitute the solid into a liquid form and achieve a liquid of a desired concentration.

The present disclosure also provides a method for forming sprayable liquids containing sensor elements useful in detecting analytes on surfaces via a sensor system as disclosed herein without requiring sampling of the surface. Further described is a process for the preparation of solid powders which can be stored for extended periods of time and at a later time dissolved, preferably with water, to reconstitute the solid powders, thus allowing for the regeneration of the enzyme-containing liquids when required. At the time of application, the solid sensor(s), indicator compound(s), and the one or more optional components selected from sensor specific substrate(s), light scattering additive(s), adhesive polymer(s), protein stabilizer(s), protein stabilizing sugar(s), surfactant(s), solvent(s) are reconstituted by adding water to each chamber of the dispensing device, drawing the water reconstituted solids (now liquids) from each chamber into a mixing area of the dispensing device for effecting a mixture of at least one or more sensor components, at least one or more indicator compounds, and, optionally one or more sensor specific substrates, light scattering additives, adhesive polymers, protein stabilizers, protein stabilizer sugars, surfactants, solvents and combinations thereof, and then delivering the mixture of the aqueous enzyme based sensor from the dispensing device to a surface, preferably through a spray head of the dispensing device. Preferably, the reconstitution of the solid materials as set forth herein is affected just prior to the application of the mixture of aqueous enzyme-based sensor to a surface.

In certain non-limiting embodiments, the trigger portion of the biomolecule can be truncated and used in the absence of the recognition portion to trigger an amplifying cascade reaction to generate a training version of the system which does not require the presence of the analyte of interest to operate. The training version can be used to teach an operator how the system would work without requiring a live analyte to be used.

### Prophetic Examples

The present disclosure will be more fully understood by reference to the following prophetic examples, which provide illustrative non-limiting aspects of the invention. It is understood that the invention described in this specification is not necessarily limited to the examples described in this section.

Example systems suitable for detecting an analyte are shown in Table 1 below:

TABLE 1

| | | | | | Connection | | | |
|---|---|---|---|---|---|---|---|---|
| | | | | | between | | | |
| | | | | Activation of | Recognition | Activation of | | |
| Exam- | Biomolecule | Recognition | Trigger | Recognition | portion and | Trigger | | |
| ple | Class | Portion | Portion | Portion | Trigger portion | Portion | Substrate | Reporter |
| 1 | Nucleotide- | Autoinhibited | Portion of | Structure | Nucleotide | Cascade | Additional | Oxidative |
| | Type | Aptamer | DNAzyme | change of | base pair | assembly of | portions | Responsive |
| | Reporter | | | recognition | binding | DNAzymes | of the | molecule |
| | | | | portion to | | | DNAzyme | |
| | | | | remove | | | and Hemin | |
| | | | | autoinhibition | | | | |
| | | | | and release new | | | | |
| | | | | nucleotide | | | | |
| | | | | binding motif | | | | |
| 2 | Nucleotide- | Autoinhibited | Self | Structure | Nucleotide | Nucleotide- | Additional | pH |
| | Type | Aptamer | Immolative | change of | base pair | templated | immolative | Responsive |
| | Reporter | | Polymer- | recognition | binding | chemical | polymers | molecule |
| | | | Nucleotide | portion to | | reaction (e.g. | responsive to | |
| | | | conjugate | remove | | Staudinger | the trigger | |
| | | | | autoinhibition | | reaction) | immolation | |
| | | | | and release new | | induces self- | | |
| | | | | nucleotide | | immolative | | |
| | | | | binding motif | | cascade within | | |
| | | | | | | immolative | | |
| | | | | | | polymer | | |
| 3 | Protein- | Antibody-type | Enzyme or | Antibody-type | Enzyme or Protein | Forced | Reagents for | Oxidative |
| | type | molecule (e.g. | Protein | binding | multi-unit | proximity | enzyme | Responsive |
| | Reporter | antibody, | complementation | induces | (2 or 3 or | reduces | reaction or | molecule |
| | | nanobody, | | proximity | 4 units - inactive | trigger | protein output, | OR pH |
| | | single- | | of trigger | when separated) | entropy to | e.g. enzyme | Responsive |
| | | domain | | elements | refolding/ | facilitate | substrates | molecule |
| | | antibody) | | | recombination/ | recombination | and/or | |
| | | | | | association | into the full | cofactors | |
| | | | | | | reporter with | | |
| | | | | | | restored | | |
| | | | | | | functional | | |
| | | | | | | activity | | |
| 4 | Protein- | Binding-type | Enzyme or | Binding-type | Enzyme or Protein | Forced | Reagents for | Oxidative |
| | Type | molecule (e.g. | Protein | molecule | multi-unit | proximity | enzyme | Responsive |
| | Reporter | glycan-binding | complementation | specific | (2 or 3 or | reduces | reaction or | molecule |
| | | proteins (GBPs or | | binding | 4 units - inactive | trigger | protein output | OR pH |
| | | lectins), | | induces | when separated) | entropy to | e.g. enzyme | Responsive |
| | | bacterial | | proximity | refolding/ | facilitate | substrates | molecule |
| | | binding proteins, | | of trigger | recombination/ | recombination | and/or | |
| | | lipid binding | | elements | association | into full | cofactors | |
| | | proteins, protein- | | | | reporter with | | |
| | | protein | | | | restored | | |
| | | interactions, de | | | | functional | | |
| | | novo-designed | | | | activity | | |
| | | binding proteins, | | | | | | |
| | | synthetic | | | | | | |
| | | polymers such as | | | | | | |
| | | molecularly | | | | | | |
| | | imprinted | | | | | | |
| | | polymers (MIPs)) | | | | | | |
| 5 | Receptor- | Autoinhibited | Enzyme or | Introduction | Enzyme or Protein | Release of | Reagents for | Oxidative |
| | Type | Receptor | Protein | of receptor | multi-unit | autoinhibition | enzyme | Responsive |
| | Reporter | | complementation | binder | (2 or 3 or | gives | reaction or | molecule |
| | | | | displaces | 4 units - inactive | confirmational | protein output, | OR pH |
| | | | | molecule | when separated) | freedom to | e.g. enzyme | Responsive |
| | | | | controlling | refolding/ | facilitate | substrates | molecule |
| | | | | autoinhibition. | recombination/ | recombination | and/or | |
| | | | | | association | into the full | cofactors | |
| | | | | | | reporter | | |

For each of Examples 1-5, the solvent can be any solvent as described herein, the analyte can be any analyte as described herein, and the measurement method can be any method as described herein. Examples 1-5 can be performed according to the method illustrated in FIG. 1 and as described herein.

### Example 6

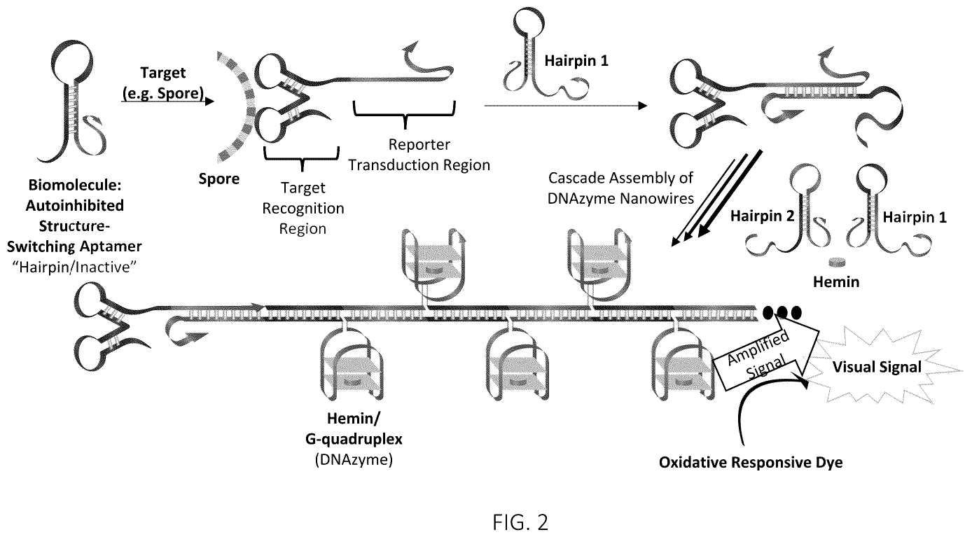

Example 6 leverages the spontaneous base pairing of aptamers to generate functional nucleic acid nanostructures capable of exponential signal amplification. Example 6 could proceed according to FIG. 2. Example 6 can target a spore analyte, use an oxidative reporter, and detect the presence of the amplified product and thus, spore analyte using a visual measurement. Example 6 utilizes recognition of analyte by the structure-switching aptamer to trigger the cascade assembly of DNAzyme nanowires from DNA hairpin reagents in solution. DNAzymes convert oxidative responsive dye into an amplified visual signal.

Upon recognition of the target DNA trigger (in this case the structure-switching aptamer) an autonomous cross-opening process yields the formation of nanowires, each containing numerous copies of the hemin/G-quadruplex. The hemin/G-quadruplex is a metal-dependent DNAzyme with functional catalytic activity mimicking that of the enzyme horseradish peroxidase (HRP). Nanowire assembly will use two functional hairpin structures 1 and 2. Hairpin 1 will consist of three-fourths of the G-quadruplex sequence at the 5′ end and one-fourth of the G-quadruplex sequence at the 3′ end, flanking regions for structure-switching aptamer recognition, and a programmed conserved sequence region for stable nanowire formation. Hairpin 2 will consist of one-fourth of the G-quadruplex sequence at the 5′ end and three-fourths of the G-quadruplex sequence (green) at the 3′ end, flanking conserved sequences. The use of hairpin structures to mask the DNAzyme sequences prevent self-assembly of the active hemin/G-quadruplex to maintain low background noise in the absence of the analyte of interest. Upon recognition of the target DNA trigger, hairpin 1 opens, exposing the conserved sequence of the nanowire backbone (red) as single-stranded DNA. Hairpin 2 is then able to hybridize to this newly available conserved region of single-stranded DNA, initiating a domino effect of hybridization between hairpins 1 and 2 for the autonomous generation of long nanowires containing the multiple copies of DNAzyme. This autonomous cross-opening process continues indefinitely, rapidly increasing the concentration of functional DNAzyme local to the analyte of interest. The HRP-mimicking function of the DNAzymes within the nanowires facilitates oxidation of chemical substrates, such as Amplex Red, ABTS, or luminol to generate an optical signal. Exponential signal amplification is derived from the continual formation of DNAzyme as well as the catalytic DNAzyme substrate turnover to achieve exceptional sensitivity.

### Example 7

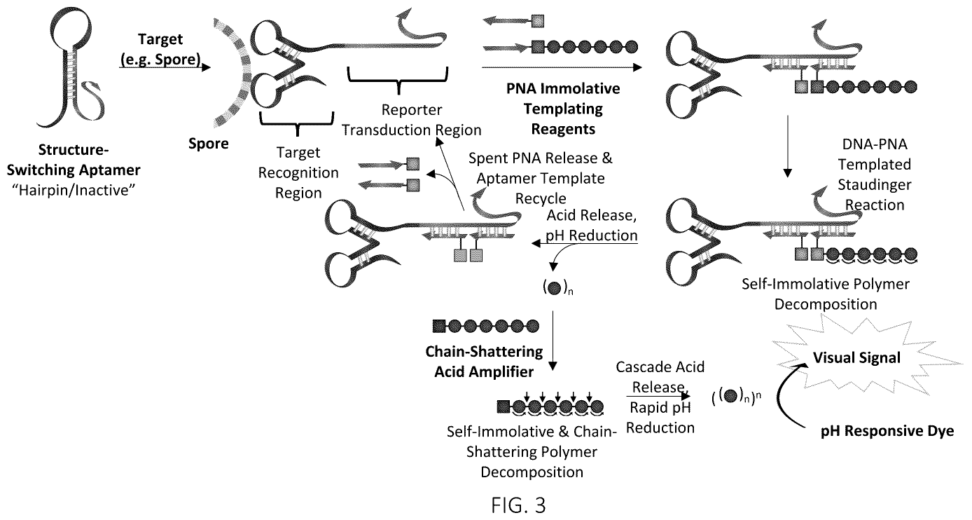

Example 7 utilizes the release of a self-immolative and chain-shattering polymer reporter system via a nucleic acid templated Staudinger reaction trigger. Example 7 could be performed according to FIG. 3. Example 7 can target a spore analyte, use a pH dye reporter, and detect the presence of the amplified product and thus, spore analyte using a visual measurement. Example 7 can recognize the analyte with a structure-switching aptamer to trigger templating of PNA polymers. Staudinger reaction initiates polymer self-immolation which can be amplified by a chain-shattering acid amplifier. The dramatic reduction in local pH could be converted to a visual signal via pH responsive dyes.

Both self-immolative and chain-shattering polymer systems are stable under ambient conditions until the reactive unit of the polymer is cleaved. Recognition of the spore analyte induces restructuring of the of the structure-switching aptamer to expose the reporter transduction region. In this case, the reporter transduction sequence facilitates a Staudinger reaction driving the chemical reaction using proximity-induced DNA templating. The Staudinger reaction acts as a trigger to initiate self-immolative decomposition of the first polymer reporter. Self-immolative polymers utilize a single triggering event to activate degradation of the entire polymer chain. The decomposition product of this first depolymerization reaction produces acid local to the target analyte. This reduction in local solution pH will trigger a second reporter which is free in solution, this time a chain-shattering immolative polymer. Chain-shattering polymers spontaneously degrade along the main chain with triggering events occurring between each monomer unit. Decomposition of the chain-shattering polymer will also produce acid but at a rate exponential to the first reporter. The change in local pH can be used to generate an optical signal through the inclusion of appropriate pH indicator dyes such as, for example, neutral red, propyl red, or methyl red. By combining the two immolative approaches, amplified chain-shattering degradation can be achieved where degradation of a self-immolative polymer generates a secondary trigger that accelerates degradation through initiation of a chain-shattering cascade.

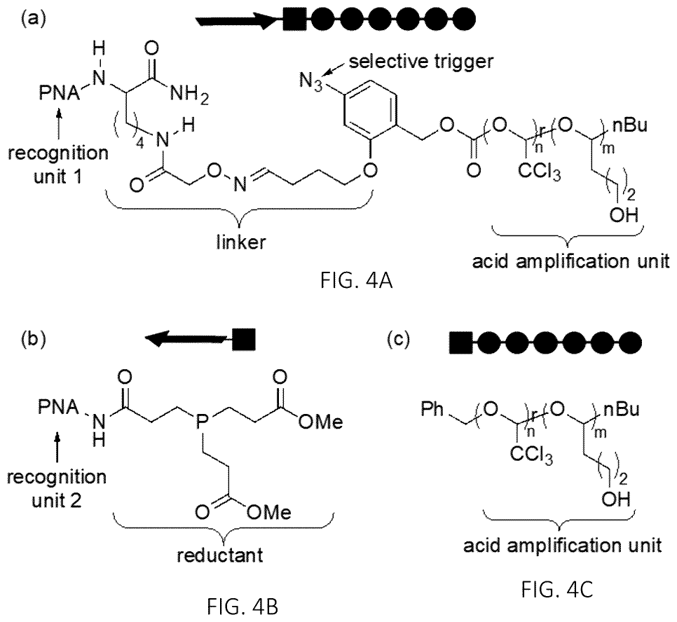

For execution of Example 7, peptide nucleic acids (PNAs) can be used as complementary strands to the structure-switching aptamer template due to their ability to base-pair with DNA and their chemical compatibility for immolative polymer synthesis. The two PNAs, each 6-8 repeating units in length, can bind to adjacent regions on the aptamer with spacing of 2-4 nucleotides between PNAs. One PNA will be linked via an oxime to an aryl azide trigger, which itself is linked to a poly(acetal) as illustrated in FIG. 4A. The second PNA can contain a pendant phosphine as illustrated in FIG. 4B. When both PNAs bind to the aptamer template, their effective concentration will increase facilitating the phosphine-mediated reduction of the aryl azide to an aniline. This type of PNA-templated Staudinger reaction occurs within minutes in aqueous systems, with essentially no background reaction in the absence of the target nucleic acid template. The third reagent can be a poly(acetal) as illustrated in FIG. 4C. The third reagent can serve the role of acid amplifier to increase the sensitivity. The templated Staudinger reduction will initiate a cascade reaction that causes release of azaquinone methide and the terminus of the pendant poly(acetal). Without an end cap, the poly(acetal) is unstable, and will depolymerize continuously, completely, and cleanly from head-to-tail within seconds to form aldehyde monomers.

FIG. 4D depicts the templated Staudinger reduction of the aryl azide to an aniline, which will initiate a cascade reaction that causes release of azaquinone methide and the terminus of the pendant poly(acetal). Without an end cap, the poly(acetal) is unstable, and will depolymerize continuously, completely, and cleanly from head-to-tail within seconds to form aldehyde monomers. The aldehyde monomers will air oxidize quickly to the corresponding carboxylic acids, which then will catalyze cleavage of the acetal backbone of any poly(acetal) in solution. This sequence of reactions will create an autocatalytic acid amplification process that will increase in rate as the quantity of acid increases in the medium. Acidification of the medium will be revealed by an appropriate choice of a colorimetric pH indicator dye.

### Example 8

Example 8 includes a reporter construct of a recognition element and an inhibitory module flanking either side of one fragment of an enzyme-type split protein reporter (split reporter A). In the “dark” state, the inhibitory module would bind the recognition element and cause either structural deformation or steric hinderance which would prevent the split reporter A from recombining with its other half (split reporter B). In this split/separate state the enzyme halves are inactive, and no signal is produced. Upon addition of the molecule of interest (MOI), the MOI would competitively bind to the recognition element and displace the inhibitory module. This displacement of the inhibitory module would release the autoinhibition of the system and relieve the conformational restriction on the split reporter A. This would facilitate a favorable conformation for recombination of split reporter A with split reporter B to generate the full reporter A/B. Recombination of the split reporter would mature the reporter structure such that the enzymatic activity is reinstated. Upon exposure to the enzyme substrate, the full reporter would turn over the enzyme substrate and generate an amplified signal.

### Example 9

Example 9 can be a specific implementation of Example 8. In Example 9, reporter fragments can be covalently attached to interacting molecules of interest (MOI) (e.g., analyte) via genetic fusion or chemical conjugation. Molecular interactions between the labeled MOIs and the target antigen drive the reporter fragments together, which, in turn, signals detection of the target antigen. Example 9 can target a spore analyte, use a split enzyme reporter, and detect the presence of the amplified product and thus, spore analyte using a visual measurement, transmittance, absorbance, Raman scattering, and/or VOC generation.

Protein complement assays (PCA) can utilize a trigger portion that is initially inactivated via fragmentation. While each fragment is inactive alone, the fragments spontaneously refold into the active reporter when brought into close proximity of one another. This proximity-induced recombination can be achieved via several types of trigger methods.

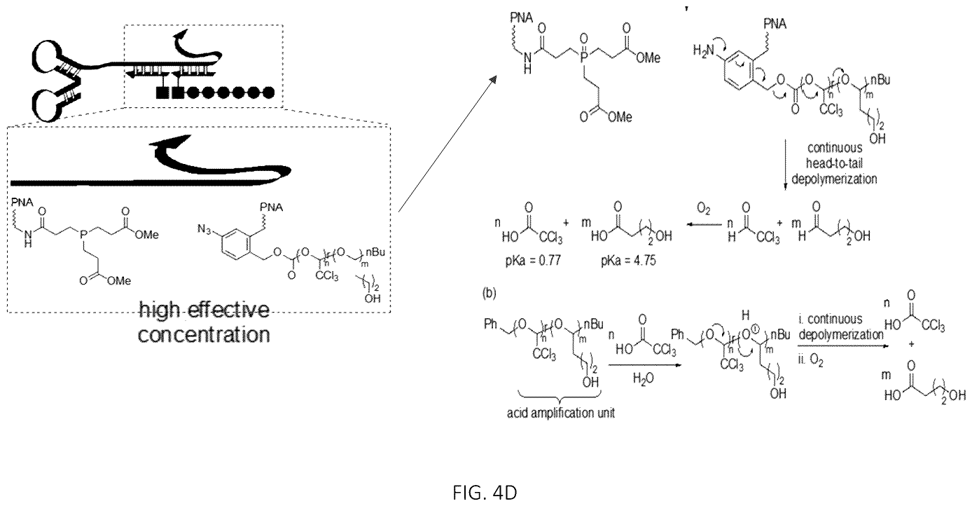

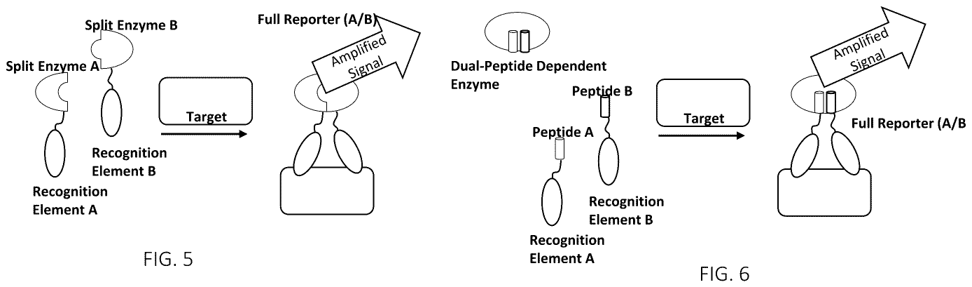

Di-part PCAs assays can exhibit a benefit in the speed of detection whereas tri-part PC assays exhibit benefits in signal specificity and in the storage stability of assay components. A Di-part PCA is illustrated in FIG. 5, and a Tri-part PCA is illustrated in FIG. 6. When used with an enzymatic reporter, such as luciferase, Antibody-Ligand Protein Complementation (ALPC) has shown promise as a versatile and scalable “add and read” antigen-based detection platform. The absolute on/off mechanism of protein complementation results in very low background signal and enzymatic amplification generates readouts have been observed by the naked eye. Other enzyme can be, for example, horseradish peroxidase, β-galactosidase, and/or green or red fluorescent protein.

### Example 10

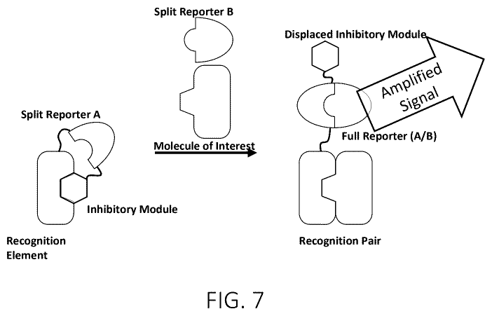

Example 10 illustrates an example of a biomolecule having an autoinhibited trigger portion that is bound to an active site of the recognition portion. Example 10 can proceed according to FIG. 7. Both the trigger portion and the recognition portion are connected to a first portion of a reporter. The first portion of the reporter is conformally inhibited while the recognition portion is bound to the trigger portion.

A second portion of the reporter is added as the substrate. When the recognition portion binds with the MOI (e.g., analyte), the first portion of the reporter is no longer autoinhibited and can bind with the second portion of the reporter and form a functional reporter (e.g., a split protein, such as a split fluorescent protein, a split enzyme, and/or a nucleotide which is was conformationally restricted and is now conformationally unrestricted and free to base pair to initiate reporting via mechanisms such as seen in FIG. 2 and FIG. 3).

### Example 11

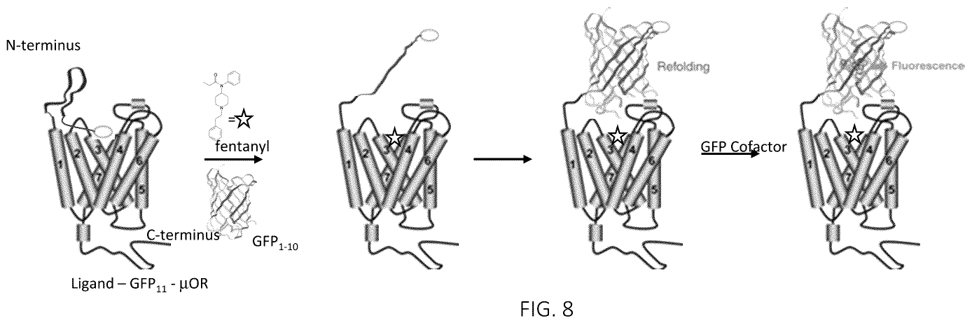

Example 11 illustrates a more specific example of Example 10. For example, in Example 11, the biomolecule having an autoinhibited trigger portion that is bound to an active site of the recognition portion can be the mu opioid receptor (μOR) bound to a portion of the split green fluorescent protein (GFP₁₁) bound to an autoinhibitory ligand for the mu opioid receptor such as the opioid peptide met-enkephalin. The biomolecule can be prepared as a tri-part fusion protein or through synthetic connection of the discrete parts (e.g. through chemical conjugation). The analyte for detection within the system can be can be an opioid (e.g., fentanyl), the recognition portion can bind with the opioid. The reporter for the system would be green fluorescent protein. Additional reagent for the system would be the remaining portion of the split protein green fluorescent protein (GFP₁₋₁₀) and the required cofactor for green fluorescent protein to be fluorescent. Switching the fluorescent protein reporter system for an enzyme (e.g. horseradish peroxidase, β-galactosidase, etc.) would convert this system into one with amplified output. Example 11 can proceed according to FIG. 8.

### Example 12

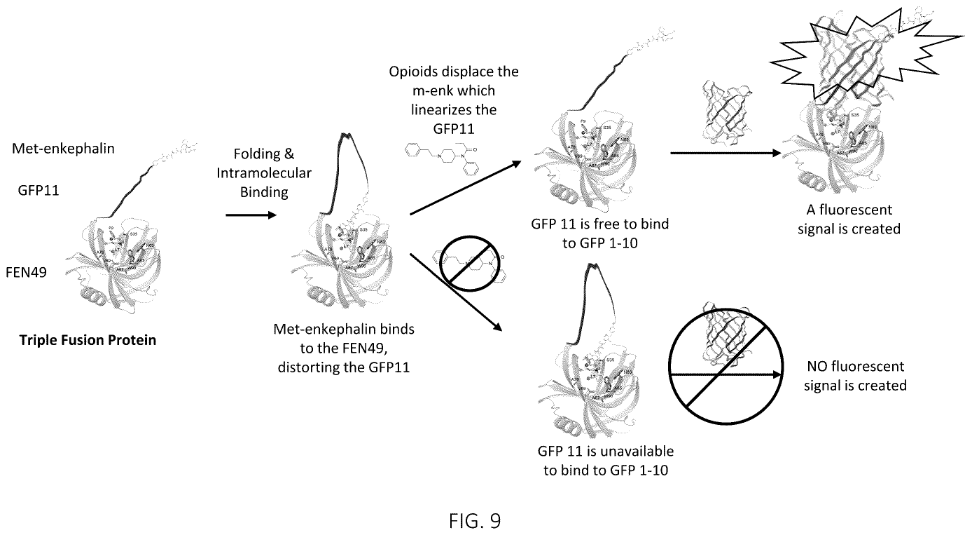

Example 12 also illustrates a more specific example of Example 10. For example, in Example 12, the biomolecule having an autoinhibited trigger portion that is bound to an active site of the recognition portion can be the synthetic fentanyl binding protein FEN49 bound to a portion of the split green fluorescent protein (GFP₁₁) bound to an inhibitory ligand for the FEN49 such as met-enkephalin. The biomolecule can be prepared as a tri-part fusion protein or through synthetic connection of the discrete parts (e.g. through chemical conjugation. The analyte for detection within the system can be an opioid (e.g., fentanyl), the recognition portion can bind with the opioid, and the reporter can be a green fluorescent protein. Additional reagent for the system would be the remaining portion of the split protein green fluorescent protein (GFP₁₋₁₀) and the required cofactor for green fluorescent protein to be fluorescent. Switching the fluorescent protein reporter system for an enzyme (e.g. horseradish peroxidase, β-galactosidase, etc.) would convert this system into one with amplified output. Example 12 can proceed according to FIG. 9.

Various aspects of the invention include, but are not limited to, the aspects listed in the following numbered clauses.

Clause 1. A method for detecting an analyte on an object, the method comprising: introducing a biomolecule to the object, the biomolecule comprising a recognition portion capable to bind with the analyte, and a trigger portion capable to participate in an amplifying cascade chemical reaction responsive to the analyte binding with the recognition portion; introducing a substrate to the object; introducing a solvent to the object, wherein the substrate, biomolecule, and solvent form a mixture on the object; binding the recognition portion of the biomolecule with the analyte; chemically reacting the trigger portion of the biomolecule with the substrate via an amplifying cascade chemical reaction to produce an amplified product responsive to the analyte binding with the recognition portion; and detecting the amplified product, thereby detecting presence of the analyte.

Clause 2. The method of clause 1, wherein the recognition portion comprises at least one element selected from the group consisting of a receptor, an antibody, an aptamer, an antigen, a bacterial protein, a nanobody, a single-domain antibody, a glycan-binding protein, a lipid-binding protein, protein-protein interaction, a de novo designed binding protein, and a synthetic polymer.

Clause 3. The method of any of clauses 1-2, further comprising introducing a reporter to the object.

Clause 4. The method of clause 3, wherein the reporter comprises at least one reporter selected from the group consisting of a pH responsive molecule and an oxidative responsive molecule.

Clause 5. The method of any of clauses 1-4, wherein the trigger portion comprises at least a portion of at least one element selected from the group consisting of an enzyme, a DNAzyme, a self-amplifying molecular assembly, and a self-immolative polymer.

Clause 6. The method of any of clauses 1-5, wherein binding the recognition portion of the biomolecule with the analyte further comprises activating the trigger portion by at least one process selected from the group consisting of proximity-based association with a second trigger portion and reduction of autoinhibition of the trigger portion.

Clause 7. The method of any of clauses 1-7, wherein detecting the amplified product comprises measuring at least one parameter of the mixture selected from the group consisting of an absorbance, a transmittance, a color, an oxidative potential, a pH, light rotation, refractive index, Raman scattering, chemiluminescence, temperature, and volatile organic chemical content.

Clause 8. The method of any of clauses 1-8, wherein the object comprises a surface of a vessel.

Clause 9. The method of any of clauses 1-8, wherein the object comprises a surface of a device.

Clause 10. The method of any of clauses 1-9, wherein introducing the biomolecule, introducing the substrate, and introducing the solvent occur simultaneously.

Clause 11. The method of any of clauses 1-10, wherein introducing the biomolecule, introducing the substrate, and introducing the solvent occur simultaneously by spraying.

Clause 12. The method of any of clauses 1-11, wherein introducing the biomolecule and introducing the substrate occur sequentially.

Clause 13. The method of any of clauses 1-12, wherein unbound biomolecule is not removed from the object prior to chemically reacting the trigger portion via an amplifying cascade chemical reaction with the substrate to produce an amplified product responsive to the analyte binding with the recognition portion.

Clause 14. A biomolecule for detecting an analyte, the biomolecule comprising: a recognition portion capable to bind with the analyte; and a trigger portion capable to participate in a amplifying cascade chemical reaction responsive to the analyte binding with the recognition portion.

Clause 15. The biomolecule of clause 14, wherein the recognition portion comprises at least one element selected from the group consisting of a receptor, an antibody, an aptamer, an antigen, a bacterial protein, a nanobody, a single-domain antibody, a glycan-binding protein, a lipid-binding protein, protein-protein interaction, a de novo designed binding protein, and a synthetic polymer.

Clause 16. The biomolecule of any of clauses 14-15, wherein the recognition portion and the trigger portion are attached by at least one of a covalent bond, hydrogen bonding, Van Der Waals forces, and a polymer.

Clause 17. The biomolecule of any of clauses 14-16, wherein the trigger portion is autoinhibited.

Clause 18. The biomolecule of clause 14, wherein the trigger portion comprises at least a portion of at least one element selected from the group consisting of an enzyme, a DNAzyme, a self-amplifying molecular assembly, and a self-immolative polymer.

Clause 19. A system for detecting an analyte, the system comprising: the biomolecule of any of clauses 14-18; and a substrate capable to react with the trigger portion of the biomolecule an amplifying cascade chemical reaction responsive to an analyte binding to the recognition portion.

Clause 20. The system of clause 19, wherein the system comprises a lyophilized powder.

Clause 21. The system of any of clauses 19-20, wherein the system is at least partially a liquid.

Clause 22. The system of any of clauses 19-21, further comprising a reporter.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification, unless otherwise stated, is intended to include all lower numerical limitations subsumed therein, and any minimum numerical limitation recited in this specification, unless otherwise stated, is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

The grammatical articles “a”, “an”, and “the”, as used herein, are intended to include “at least one” or “one or more”, unless otherwise indicated, even if “at least one” or “one or more” is expressly used in certain instances. Thus, the foregoing grammatical articles are used herein to refer to one or more than one (i.e., to “at least one”) of the particular identified elements. Further, the use of a singular noun includes the plural, and the use of a plural noun includes the singular, unless the context of the usage requires otherwise.

One skilled in the art will recognize that the herein described systems, methods, structures, operations/actions, and objects, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class, and the non-inclusion of specific components, devices, apparatus, operations/actions, and objects should not be taken as limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

What is claimed is:

1. A method for detecting an analyte on an object, the method comprising:
introducing a biomolecule to the object, the biomolecule comprising
a recognition portion capable to bind with the analyte, and
a trigger portion capable to participate in an amplifying cascade chemical reaction responsive to the analyte binding with the recognition portion;
introducing a substrate to the object;
introducing a solvent to the object, wherein the substrate, biomolecule, and solvent form a mixture on the object;
binding the recognition portion of the biomolecule with the analyte;
chemically reacting the trigger portion of the biomolecule with the substrate via an amplifying cascade chemical reaction to produce an amplified product responsive to the analyte binding with the recognition portion; and
detecting the amplified product, thereby detecting presence of the analyte.

2. The method of claim 1, wherein the recognition portion comprises at least one element selected from the group consisting of a receptor, an antibody, an aptamer, an antigen, a bacterial protein, a nanobody, a single-domain antibody, a glycan-binding protein, a lipid-binding protein, protein-protein interaction, a de novo designed binding protein, and a synthetic polymer.

3. The method of claim 1, further comprising introducing a reporter to the object.

4. The method of claim 3, wherein the reporter comprises at least one reporter selected from the group consisting of a pH responsive molecule and an oxidative responsive molecule.

5. The method of claim 1, wherein the trigger portion comprises at least a portion of at least one element selected from the group consisting of an enzyme, a DNAzyme, a self-amplifying molecular assembly, and a self-immolative polymer.

6. The method of claim 1, wherein binding the recognition portion of the biomolecule with the analyte further comprises activating the trigger portion by at least one process selected from the group consisting of proximity-based association with a second trigger portion and reduction of autoinhibition of the trigger portion.

7. The method of claim 1, wherein detecting the amplified product comprises measuring at least one parameter of the mixture selected from the group consisting of an absorbance, a transmittance, a color, an oxidative potential, a pH, light rotation, refractive index, Raman scattering, chemiluminescence, temperature, and volatile organic chemical content.

8. The method of claim 1, wherein the object comprises a surface of a vessel.

9. The method of claim 1, wherein the object comprises a surface of a device.

10. The method of claim 1, wherein introducing the biomolecule, introducing the substrate, and introducing the solvent occur simultaneously.

11. The method of claim 1, wherein introducing the biomolecule, introducing the substrate, and introducing the solvent occur simultaneously by spraying.

12. The method of claim 1, wherein introducing the biomolecule and introducing the substrate occur sequentially.

13. The method of claim 1, wherein unbound biomolecule is not removed from the object prior to chemically reacting the trigger portion via an amplifying cascade chemical reaction with the substrate to produce an amplified product responsive to the analyte binding with the recognition portion.

14. A biomolecule for detecting an analyte, the biomolecule comprising:
a recognition portion capable to bind with the analyte; and
a trigger portion capable to participate in a amplifying cascade chemical reaction responsive to the analyte binding with the recognition portion.

15. The biomolecule of claim 14, wherein the recognition portion comprises at least one element selected from the group consisting of a receptor, an antibody, an aptamer, an antigen, a bacterial protein, a nanobody, a single-domain antibody, a glycan-binding protein, a lipid-binding protein, protein-protein interaction, a de novo designed binding protein, and a synthetic polymer.

16. The biomolecule of claim 14, wherein the recognition portion and the trigger portion are attached by at least one of a covalent bond, hydrogen bonding, Van Der Waals forces, and a polymer.

17. The biomolecule of claim 14, wherein the trigger portion is autoinhibited.

18. The biomolecule of claim 14, wherein the trigger portion comprises at least a portion of at least one element selected from the group consisting of an enzyme, a DNAzyme, a self-amplifying molecular assembly, and a self-immolative polymer.

19. A system for detecting an analyte, the system comprising:
the biomolecule of claim 14; and
a substrate capable to react with the trigger portion of the biomolecule to produce an amplifying cascade chemical reaction responsive to an analyte binding to the recognition portion.

20. The system of claim 19, wherein the system comprises a lyophilized powder.

21. The system of claim 19, wherein the system is at least partially a liquid.

22. The system of claim 19, further comprising a reporter.
